(19) United States
(12) Patent Application Publication
(10) Pub. No.: US 2023/0248270 A1
(43) Pub. Date: Aug. 10, 2023

(54) AUTONOMOUS BIO-POWERED NANO DEVICES FOR IMPROVING HEALTH AND QUALITY OF LIFE

(71) Applicant: Richard Postrel, Miami Beach, FL (US)

(72) Inventor: Richard Postrel, Miami Beach, FL (US)

(21) Appl. No.: 17/609,108

(22) PCT Filed: Jun. 24, 2021
(86) PCT No.: PCT/US2021/039014

§ 371 Date: Nov. 5, 2021

Related U.S. Application Data
(60) Provisional application No. 63/043,297, filed on Jun. 24, 2020.

(51) Int. Cl. A61B 5/145; A61B 5/024; A61B 5/0205

(52) U.S. Cl. CPC A61B 5/14532; A61B 5/14546; A61B 5/14542; A61B 5/02444; A61B 5/02055; B82Y 15/00

(57) ABSTRACT

The present invention provides systems, methods, and devices for autonomous bio-powered medical management and reporting. The systems allow for interactivity between multiple devices for various activities including, but not limited to: medical diagnostics, therapeutic modulation, recording, and reporting. One or more of the autonomous bio-powered devices can be wearable and/or implantable (e.g., subdermal, spinal, cranial, pulmonary, musculoskeletal, urethral, etc. monitoring and control). These can replace, with a continuous stream of current status reports: thermometers, pulse oximeters, CO₂ monitors, local circulation, local, regional or orgasmic nutrition status, cardiac rate and output, glucose, pharmaceutical levels, electrolytes, balance, individual muscles, muscle systems, sensory inputs, auditory inputs, visual inputs, etc., to arrive at a capability for delivering modulating treatments and/or induce mechanical, medical and/or physiological responses.

The present invention provides systems, methods, and devices for autonomous bio-powered medical management and reporting. The systems allow for interactivity between multiple devices for various activities including, but not limited to: medical diagnostics, therapeutic modulation, recording, and reporting. One or more of the autonomous bio-powered devices can be wearable and/or implantable (e.g., subdermal, spinal, cranial, pulmonary, musculoskeletal, urethral, etc. monitoring and control). These can replace, with a continuous stream of current status reports: thermometers, pulse oximeters, CO₂ monitors, local circulation, local, regional or orgasmic nutrition status, cardiac rate and output, glucose, pharmaceutical levels, electrolytes, balance, individual muscles, muscle systems, sensory inputs, auditory inputs, visual inputs, etc., to arrive at a capability for delivering modulating treatments and/or induce mechanical, medical and/or physiological responses. The present invention relates to mankind's increased attention to fitness and health. The physical, chemical, biological and medical sciences have seen many advances as mankind has discovered other cultures, traveled, increased abilities and modes of communication with a result that general tenets and discoveries such as gravity, inorganic and organic chemistry, and the genetic bases underlying large and small creatures and microscopic pathogens are understood on general and increasingly almost tedious details. With the computer age and massive ability to collect and analyze data, we are finding that more and more specific details can be a step in improving many types of outcomes.

A large part of this improvement is attributable to the development and use of the computer to manage, store and analyze massive amount of data. The ability to gather specific data to be incorporated into the larger knowledge base allows individual assessments to both contribute and benefit from computer learning skills applied across large databases.

Following the publication of the human genome and the commercialization of gene sequencing pursuits for risk analysis or personal interests, the personalization of medicine is a growing field of interest. Better diagnostic tools can aid clinicians in improving patient outcomes. Many developments are contributing to these efforts including, but not limited to immense data storage capabilities in multiple interconnected sites and robust analytical computing machinery to apply the data. One aspect of these hoped for advances applies the process of machine learning to notice patterns and to associate these patterns with particular traits, diseases, treatments, etc. Thus, personalized medicine has 2 important and seemingly opposite aspects. First, at the individual level, every nuance that distinguishes the person and defines the individual must be considered if “personalization” is to be real. Second, massive stores of data from the environment, and from other individuals (sometimes non-human animals) are necessary for the machine learning components to reference in order to propose and confirm patterns for comparison against an individual's particular presentations. The “individual” then is modeled into the system (possibly anonymously) across multiple levels (e.g., cell, relevant drugs, organ, circulation, nutrition, etc.). The artificial intelligence component then runs multiple scenarios, much like financial advisors commonly do, to hopefully arrive at a small number of multi-step treatment protocols with predicted or expected results of each intervention and suggested corrections when the results deviate from expectations. Long story, short: Personalized medicine should provide more predictable and better treatment outcomes for each participant patient, but massive stores of personal data and background societal data for smart comparison are required for this dream to progress. Devices of the present invention are a valuable component in this process.

Dynamic data collected repeatedly from a single individual are also important for optimal personalization. As one example, our bodies follow a daily cycle including sleep and activity cycles and learned cycles when our organs expect food. Adenosine, melatonin, sugars, insulin, glucagon, MSG, adrenocorticotropic hormone, cortisol, luteinizing hormone, gonadotropin-releasing hormone, follicle stimulating hormone, dopamine and other neurotransmitters, etc., cycle throughout the day. Monthly cycles and seasonal cycles are also factors to be considered. Personalized medicine, accordingly, cannot rely on a single snapshot to get a true picture of the individual, but must consider the individual's dynamic responses. For example, diabetics benefit from multiple tests and possible interventions throughout a day. Smart watches already externally sense and may collect data for a range of data including, but not limited to: blood oxygenation, CO₂, pulse or heart rate, temperature, sleep cycling, tissue degradation, microbiome secretions and breakdown products, compounds correlated with a specific disease (e.g., C-reactive protein, renin, angiotensin, aldosterone, disintegrin, epinephrine, etc.) compounds associated with a class of diseases (e.g., cancers, viral diseases, etc). For example caspase activities correlate across e.g., RNA viral infections and many cancers. Personalized medicine considers not just the circulating caspase residues, but associates these data with multiple other readings from the person. For example, the altered caspace findings in conjunction with circulating sFas fragments and/or its ligand FasL focuses on oncologic disease rather than viral. Other circulating markers left as byproducts of apoptosis or necrosis or compensating responses to the cell death may be apparent as cytokeratins, circulating DNA fragments, Bcl2, increased angiogenetic stimulating factors, FGF, etc. may be combined with detected cell specific residual fragments to identify the target of a viral attack and/or the underlying cancerous tissue source. The multiple nanosensors in various available arrays simplify these personalizations by detecting many multiples of biologic markers and events and using artificial intelligence concentrating on specific patterns associated with one or more diseases or conditions of the individual.

The present invention massively expands the group of available parameters by incorporating a large variety of an individual person's data into a compendium of hundreds, thousands, or millions of person's individualized data. Then, using this database or portions thereof, each contributing individual can benefit from real time analysis and pattern recognition to apply lessons learned to any individual's ultra-specific circumstance.

For devices used in a sensor mode, data transfers to an external transponding or recording device are available using any means known in the art, for example, a wearable or other configured device, e.g., a 5G device, a wireless server, an intranet or internet server associated receiver may be in continuous or intermittent interface with the internal sensors. Recharge windows are available to correspond to needs of the various functions. For example, in a low power consuming sensor function without significant power consuming analysis or computation, a charge may be scheduled, e.g., wirelessly every 180 days. As consumption requirements exceed e.g., perhaps 1 μwatt, a more frequent interface may be accomplished for power and/or data/information transfers. A morning or evening charge such as shower or mealtime may be preferred in some embodiments; an overnight charge/data transfer may be preferred in some embodiments, either removing the wearable for charging or charging in situ; a continuous or available on demand interface device such as a band may be worn long-term, including continually. Data transfers and power transfers may occur coincidentally, may be staggered, may be coincident only for brief segments with e.g., the charging period being in excess of a transfer period.

The nano-crafted sensors of the present invention are provided to enable massively expanded pools of available information relating to an individual's internal metabolisms and to share these data for enhanced pattern recognition across individuals. Sensors are provided for general or specific purposes and positioned under the skin or at predetermined sites of interest for one or more diseases or traits in question. Sensors may be crafted to provide data relating to a single characteristic, e.g., a nutrient concentration near a selected organ, local blood flow, blood oxygenation, etc. Designed features for data collection including, but not limited to: 02, CO₂, pH, sugar, temperature, proteins in general, a specific protein, a molecule bound to or transported by a protein, osmotic strength, salts, hormones, stimulating factors, releasing factors, inhibiting factors, drug, drug metabolites, % lipids, organic compounds, cytokines, inflammation, allergic responses, blood flow (including cell counts and type of cell), lymph, movement, etc. Internalized nanosensors communicate remotely with external devices for compilation, further analysis and possible alert for a relevant condition, such as: loss of attentiveness (e.g., α-sleep for machine operator), an impending seizure, conditions leading to an asthma attack, early migraine and other auras, etc. Such alert may be private to the individual or may be shared with another machine or person, for example a health monitoring service or clinician. The communication may be two-way with the external device providing additional instruction to the nanosensor device. The external partner may be a dedicated device, such as a ring, wrist band, ankle band, belt, etc., or may be a general multi-function device such as a smart watch or smart phone. Sensor elements will generally sense biologic information. However, sensor elements may function as receivers of information, for example, instructional information, when designed as actuation components. Actuation components may chemically or biologically modulate bio-function or may electrically stimulate or relax bioactivity. Portions of sensors and auxiliary components are preferably coated with an inert material such as parylene to minimize fouling which may bias sensitivities and to serve as a barrier against attack by the immune system.

An actuation component integral with or spatially separated from said biologic information sensing element when notified of underlying pre-symptomatic events that may develop into a relevant undesired condition reacts according to program instruction to reduce, delay, modulate or prevent said condition, for example by releasing or stimulating release of a neuro-receptor agonist or antagonist, stimulating (electrically, physically or biochemically) at least one cell to provide release or slow including slowing to zero release of a stimulant or inhibitor. For example, a neurotransmitter (including, but not limited to:

amino acids (such as glutamate (aka MSG), aspartate, D-serine, γ-aminobutyric acid (GABA) and glycine), nitric oxide (NO), carbon monoxide (CO), hydrogen sulfide (H₂S), dopamine, norepinephrine, epinephrine, histamine, serotonin (5-HT), phenethylamine, N-methylphenethylamine, tyramine, 3-iodothyronamine, octopamine, tryptamine, oxytocin, somatostatin, substance P, endogenous opioids, adenosine triphosphate (ATP), adenosine, dopamine, acetylcholine (ACh), activating or suppressing endogenous cannabinoids, (e.g., virodhamine, anandamide, N-arachidonoyl 1-serine, N-arachidonoyl dopamine, 2-arachidonoylglycerol, etc.) or transmitter action might be stimulated or blocked, e.g., by its natural agent or a surrogate drug such as caffeine. Natural or artificial endocannabinoids when released in proximity to the desired site of action are a broadly applicable modulation tool, sometimes calming (e.g., caffeine on adenosine receptors, N-arachidonoyl 1-serine reversing virodhamine induced chemotaxis but on different cannabinoid receptor molecules, but sometimes exacerbating the undesired precursor events. Opposing cannabinoids are most effective, sometimes solely effective, in the presence of the cannabinoid whose action they oppose. Accordingly, sensitive local assays increase the ability to effectively direct release (either from storage in an actuation unit or by stimulating release by proximal cells). The sensor element through sharing information with machine learning may be intelligently programmed to recognize and respond to the specific individual's biology, time of day, stress indicators, etc. to optimize corrective actuations. Internal computer (micro controller) normally operating in quiescent low power mode, wakes upon schedule or in response to event or receiving a signal

Nano-sensor technology has benefited many fields including, but not limited to: industrial toxins, quality control, security, health care, etc. Along this path, for example, Hitachi markets a micro-sensor (slightly larger than nanosensor) 128 bit ROM chip of 150 μm×150 μm×7 μm implantable chip. Micro-electromechanical nano-micro-systems (MEMS) can detect everything from light to vibrations to temperature. Chemo-sensors have been incorporated into nano-sensors for peptides, proteins salts, small organics, ions, liquid organic chemicals, volatile organic chemicals, radiation, light, etc.

Sensors of the invention thus may monitor attributes associated with biological activities including, but not limited to: volatilizable organic compounds, pulse, temperature, Reynaud's (e.g., temperature, circulation, ischemia, gangrene, oxygenation, etc.), glucose, hormones (e.g., melatonin, serotonin, thyroxin, triiodothyronine, epinephrine, norepinephrine, dopamine, anti-müllerian hormone (AMH), adiponectin, endorphins, pH, local concentration of a chemical messenger, oxytocin, nutrient level, 02, vitamin, cell count, size, ionizing radiation—e.g., chemotherapeutic drugs/metabolites, fatty acids, clumps-platelets, etc., agglomerated proteins, pressure (e.g., from tumor), density (with sonar), nerve velocity, responses, feed power to chips for hearing enhancement, enhance or dampen nerve signal, imbalance sensor, alcohol, pineal control, adenosine, melatonin, cannabinoid response, epileptic warning/control, migraine aura or pre-aura, immune activities, cytokine block/enhance activators, seizures, muscular control—tighten bladder pressure and release, esophageal function, regurgitation, acid reflux, hiccups, nervous ticks, Touretts, sense and intercept migraine or seizure aura, block or warn, osmotic strength ionic strength, individual ions, adrenocorticotropic hormone (ACTH), follicle stimulating hormone (FSH), thyroid stimulating hormone (TSH), thyroid releasing hormone (TRH), cholecystokinin (CCK), gastric inhibitory polypeptide (GIP), corticotropin-releasing hormone (CRH), prolactin releasing hormone (PRH), bone gamma-carboxyglutamic acid-containing protein (BGLAP), glanin, renin, growth, luteinizing, oxytocin (OXT), human chorionic gonadotropin (HCG), etc.

Using this astounding amount of sensing and storage capacity packed into a miniaturized volume, MEMS chips internally combine selected sensing and computing that is supported by an autonomous power supply and wireless communication capabilities in a minuscule space, typically at a scale of a few mm³ in total volume. Thus, MEMS are used to: collect various data types including, but not limited to: acceleration, stress, pressure, humidity, sound, etc.; process the data with onboard computer capability; store raw and/or processed data in memory; and wirelessly communicate select data to a local computer device, the cloud and/or linked MEMS.

The intended target for data collection will determine the size, shape, implantation method, implantation location, etc. of the nanosensor. Larger sized packets or capsules, e.g., for ease of surgical implantation and securing at the desired site may be crafted. These larger sized packets may share architectural designs of more intimate nanosensors, being manufactured at an atomic or molecular scale. The implantable devices will be designed with particular needs in mind, such as impact resistance for needleless injection, size, etc. To maintain functionality the primary power source of the present invention derives from the individual's own bodily functions. A preferred source is the mechanical energy evident in normal activities, such as moving, walking, breathing, etc. Data collection can be designed to require minimal power and thus stress or modulation of normal human function. At times some features of the present invention may benefit from additional power that may be provided to internal devices from autonomous generation modules or from external transponders.

For example, two-way communications may comprise simply data and instructions, but may also benefit from increased bandwidth and data density using an enhanced or secondary powering system. Some embodiments may prefer that data analysis be performed internally, i.e., before transmission to the supporting external database. Thus, some embodiments may include one or more auxiliary external devices for energizing functions of the internal sensor or activator module. Powering a sensor chip requires reliable electrical energy, including an energy storage bank such as a battery, capacitor, micro-fuel cell, etc. Feeding this storage device, hereinafter often generalized as “battery” for convenience, requires an energy transducer feeding off some kind of gradient. Small size requires extreme miniaturizations for all functions to reside in a module preferably much smaller than a grain of rice. For example, a voltage gradient is maintained in many cells with a Na⁺/K⁺ ATPase. Flow would be a possible source with an electrolyte (blood) pulsing an electric field. But the microchip would not reliably be stationed next to a pulsing blood vessel.

A primary battery would need a low power drain, a high power density, and adequate size to work. Such is not generally feasible for the volume and length of activity envisaged in the present invention. A primary battery would lose charge rendering the device dead and useless when the charge dissipated. A secondary battery has advantages in that continual or constant re-charging can restore and maintain long-term device functionality. When normal physical-mechanical-chemical activities might not suffice for robust functions of the internal devices, an external booster, such as built into a brace, a band, a pillow, etc. may serve as a secondary booster source for recharging, preferably fully charging the device at intervals when such external transducers are convenient.

One primary powering source features a miniaturized mechanical movement providing energy to be transduced into a stored electric or chemo-electric bank. A micro generator is craftable in a miniature Micro Electrical Mechanical System (MEMS) using mechanical motion. The motion may be provided by a person's pulse driving a mechanical pump. When walking, muscle contraction provides a different pulsating motion. The motion be directly converted to electrical energy or an intermediary, such as a spring may be wound and released at timed or threshold intervals to charge a secondary battery or a capacitor for computation and transmission. One form is based on a “Gyroscope-based electricity generator” as described in U.S. Pat. No. 7,375,436: “For example, the motion of water waves in a large body of water, e.g., lakes, rivers, and oceans, may be used to generate electricity. Oceans, in particular, have an enormous potential as a source of energy in part because oceans cover over 70% of the earth's surface and are estimated to have an annual capacity of about 2000 tera watt-hour in the surface wave energy alone.” These systems are proven in principle and thus when modified for the nano fabrication and disposition environment are one available power source for supporting the presently disclosed system.

A Seebeck effect is another available energization source. Body heat may participate as part of a thermoelectric generator (TEG) system. TEGs are sometimes referenced as Seebeck generators. TEGs of the present system would preferably be configured as solid state devices that convert heat flux (temperature differences) directly into electrical energy through the phenomenon known by the term “Seebeck effect” (one form of thermoelectric effect). One patent referencing such is titled “INCREASING THE SEEBECK COEFFICIENT OF SEMICONDUCTORS BY HPHT SINTERING” U.S. Pat. No. 8,394,729 which instructs: “Measurements of the Seebeck effect are reported as the Seebeck coefficient. The Seebeck effect, or the thermoelectric effect, is the voltage difference that exists between two points of a material when a temperature gradient is established between those points.” . . . . For instance, bismuth telluride (Bi₂Te₃) and lead telluride (PbTe) are two commonly used semiconductor thermoelectric materials with optimized Seebeck coefficients greater than 200 μ/K.”

Internal power may also be generated using nanowires, e.g., bio-nanowires with electrical-generating functions in the presence of water. Several bacterial species produce extensions off their membranes to form electrically conductive protein spikes. A thin film protein nanowire device has been developed from Geobacter sulfurreducens, one of the bacterial species so far recognized as expressing these “pili”. One proven application is the continuous generation of electric power using only vapor phase water present in atmospheric moisture. Thin film (7 micron) nanowire from the pili proteins have been effective in generating a continuous electric current from a 0.5 volt gradient with a current density measured at 17 μamperes/cm². Stacks of such slabs may be used to generate higher voltages. Multiple depositions of such stacks, e.g., in a rolled cylinder, can be used to increase the available current. A semi-permeable membrane, e.g., one permitting passage of water vapor but not the liquid, when used in conjunction with internalized devices of the invention can provide continuous power without external energy input. The power may be stored in an associated battery or capacitor to support repeated spikes of signaling from the implanted device to an external receiver. The Geobacter sulfurreducens is an example of microbes known to express these nanowires. Several other species and variants are known with additional microbes constantly being recognized with similar traits.

A preferred system allowing more expeditious modifications may involve remote wireless electronic charging. An external charging source can serve as a primary or as a reserve power provider or booster to provide energy to the embedded device. The external source may be tasked with providing power to the internal components at times of extreme demand, such as packeted data transfers, may continuously or on activation provide power, or may serve as a an augmentation or back-up power provider when the internal generator proves insufficient for demand. Using alternating current in an induction charging capacity is one such embodiment. However, energy transfer is also possible using electromagnetic or acoustic energy with paired sender-receiver devices. Such integrated system may be multi-channel, e.g., sporting at least one channel for inductively charging one or more secondary batteries while carrying at least a second channel configured as a communicating wave.

A remote charging system, i.e., one not in direct physical contact with an internal device is an especially robust option for higher power consuming implants. Induction has been gaining in popularity as a charging or power source. A chip carrying a receiver can receive and acknowledge a pulsed signal. Signals should comprise two-way encryption to block external hacking and data theft. Directionality and signal strength can be additional factors to eschew hacking. Error detection and one or more correction algorithms appropriate for the data use and reliability requirements or as required by industrial or regulatory standards should also be included. The acknowledgment can be a short or long stream of data and given its digital nature is easily encrypted. Simple versions of such devices are used as implants in pet identification chips. Larger transponders either linear or coiled can transpond over greater distances or are more readily charged. External transponders can both be used as energy providers and as data receivers for acknowledgments and/or data transmissions from implanted devices. External wearables may carry their own power packs but may also take advantage of kinetic movements to self-energize and induce energy in the implant. Appropriate wearables can be of any design, glasses, headbands, armbands, belts, jackets, sweaters, socks, shoes, leg coverings, etc., and may themselves generate (transduce) their own power or may have all or a portion provided by another source. Such wireless transfers are capable of deep penetration with efficiency ˜70% using 20 mm surface coil penetrating to 100 mm through tissue.

A remote charging system can itself include generator function, e.g., one or more of a Seebeck generator, a solar or ambient light powered generator, a mechano-electric generator, etc. Embodiments may include wearables including, but not limited to: a band (joint band, headband, fashion statement band), a pillow, a watch, etc. Such band or band surrogate may supply charge to one or more implanted devices through auto-generation capacities through acting as an energy transducer, or perhaps as a long term, high capacity, charge depository providing additional charge (and potentially transferring data and or instruction to an implant). A preferred integral charging system may comprise an electromagnetic generator tuned to resonate with a subject's walking or biking frequency, or may resonate to coordinate with typing motions, breathing timing, etc. frequency. The periodic or cyclic regular motions are converted into an electric wave that is used to feed a storage device such as a battery, preferably solid state in some embodiments, or capacitor.

A Piezo electric membrane deformation generation system is another option. Carbon nanotube sensors can be designed to exhibit low energy consumption in the pico to femto amp range. Analogous structures including, but not limited to: graphene based (planar, curved, crumpled, etc.), Si containing (including SiC), nitride containing (including AlGaN/GaN, SiₓN_{y}, GaN, graphitic carbon nitrides, BN—e.g., hexagonal BN, aluminum nitrides, with similar energy consumption can be incorporated. See e.g., “Piezoelectric fluid-electric generator” U.S. Pat. No. 4,387,318 and specifically: MECHANICAL TO ELECTRICAL TRANSDUCER DEVICE U.S. Pat. No. 5,380,1839 which teaches: “In addition to single crystal Rochelle salt, quartz, lead zirconate-titanate porcelains etc., there have been previously discovered synthetic high molecular compounds specially processed as piezoelectric materials. These synthetic high molecular compounds have been utilized in the form of film, such as polyvinylidene fluorides, polyvinyl chlorides, polycarbonates, 11-nylon etc. It is well known that films of such high molecular compounds can be rendered considerably high in piezoelectric properties by elongating the film maintained at a temperature adjacent to the softening point thereof, applying a DC voltage across the film in the direction of thickness thereof and raising the temperature of the film while the DC voltage continues to be applied thereacross.” Several of the materials that may serve as a base for doping to improve selectivity and/or sensitivity may be similarly doped for energy harvest and transduction.

A μwave transmission in a range not absorbed by human tissue (including the proteins, fats, water, etc. therein) is another potential source for remote powering and/or information transfers. Microwaves, though used for a variety of unrelated tasks such as cooking and line-of-sight remote communication, are spread over an extensive bandwidth. In general, microwaves are defined to have wavelengths of 1 mm to 1 meter with corresponding frequencies of 300 GHz-300 MHz. Consumer microwave ovens cook with a normal 2.45 gigahertz (GHz) or 12.2 cm (4.80 in) frequency and wavelength. This sub-band and several other sub-bands are absorbed by certain molecular materials and thus are preferably avoided for communication or charging duties. Other non-biologic wavelengths may have restrictions in one or more countries so as to avoid convention, law or regulations relating to their communication uses.

An externally worn device may serve as an auxiliary sensor or serve in a supportive role. For example, such auxiliary device may be used to confirm calibration or participate in re-calibration of the internal device. The auxiliary may provide information relevant to analysis of the internally sensed data, including, but not limited to: ambient light status, temperature, barometric pressure, etc. For example, a wrist band, a knee band, a ring, a bracelet, an anklet, a shoe insert, a body piercing, a strap, etc., may be worn during part of a day or with specific activities or may be worn almost permanently or continuously. These external devices may share sensing characteristics with the internal sensing device(s), for example, the external band, strap, ring, etc. may assay volatile organic compounds (VOCs) emitted through the skin while the internal sensing may assay these compounds dissolved in body fluids. The VOCs may be identical or chemically related, for example depending on the interaction of the compound with water.

Devices in a modulator mode may be pre-programmed to react to signals for an associated sensor. Modulator modes will generally be activators or inhibitors, such as secretors or hormones, local nutrition, receptor agonist or antagonist, apoptosis inducer, apoptosis inhibitor, electrical pulse to dampen or agonize local or regional pulses, etc.

Especially in the modulation mode, the self-generation and/or wireless charging embodiments avoid active pathways through the skin. The skin and its protective functions are intact and continued as the internal functions as proper for the need, such as pain management, sense restoration or replacement (e.g., cochlear implant, spinal cord implant, brain stimulation—reticular activating system, pineal, vagal stimulation, etc.), a control of electrical pulsation (e.g., a seizure), chemical stimulation or inhibition, increased growth, data collection, etc., proceed. Implants may be designed for various sites and functions. Thus, an implant may be simply subdermal or intramuscular. Deeper implants are possible, e.g., spinal, cranial, pulmonary, urethral, bladder, etc. Implant functions can replace, with a continuous stream of current status reports medical devices such as: thermometers, pulse oximeters, CO₂ monitors, local flow or circulation monitors, local, regional or orgasmic nutrition status, breathing rate, inspiration/expiration flow volume, cardiac rate and output, glucose, pharmaceutical levels, electrolytes, balance sensations, individual muscle movement, muscle systems and coordination, etc. One or more devices may monitor or provide sensory inputs, e.g., movement, tactile sense, vibration, temperature, auditory inputs, visual inputs, etc. Charging, data transfer, instructional transfers, modulation functions are all available without additional windows through the skin.

In medicine, possible applications are immense especially from providing specialized diagnostic procedures with only minimally invasive interventions without heavily invasive surgery. As designed and programmed for in home monitoring the device may be personally programmed to alert a medical professional or designated contact when e.g., predetermined or unexpected events are evident. Drug use relapse may be one of the pre-programmed alerts. The device may be used in conjunction with an aide visit, for example, providing guidance to the aide relating to questions to ask the subject, suggesting physical therapies to encourage proper individualized movement, suggesting meals or nutritional supplements. The device may be used to reveal prevarication such as a bulimic person not mentioning purge events. The device also may help to mitigate skilled health aide shortages by improving efficiency of patient-aide interactions. The personalization also may be nuanced to simply monitor a person's whereabouts. For example, using a 5G connection, the device may keep tabs on a person exhibiting dementia. In a similar application the device may be used for security monitoring, e.g., to geolocate a person under house arrest or other restrictions such as a restraining order. Device removal would be apparent because the personal physiology of the assigned device bearer would not be duplicatable, even for identical twins. In a similar security application the device may be pre-programmed to prevent operation of a machine or motor vehicle when the subject person is, e.g., in a light sleep phase, intoxication, outside a permitted area, etc.

Breakthroughs in silicon and fabrication technologies, allow targeted miniaturization down to about the size of a grain of sand which would be designed to contain nano-micro-sensors, computing analysis, sending and receiving wireless communications and a power supply in an isolated unit. Because of this incredible miniaturization, both nanochips and Smart dust have the capacity to infiltrate the human body, become lodged within, and begin to set up a synthetic network on the inside which can be remotely controlled from the outside. For example, as long ago as 2015, IBM was producing nanochips in close to bio-molecular size (˜3× the width of a DNA strand), only about 7 nm in size.

Miniaturization continues to improve. Although the 2015 chip did not incorporate its own power supply, it possessed powerful computational ability for use inside a device when connected to an int and out-put transducer and a power supply.

Sensing inputs are now available using nano-technology/chips that can be interfaced with or incorporated into such computational chips. Incorporation allows more refined miniaturization.

An ionic solution moving in a pulsing fashion induces an electric field. A device in proximity to such field can apply the pulsing field to generate electric power. Mechanical pulsing from, for example, any active body movement—e.g., walking, swinging arms, blood flow and blood pulsing, muscle movement, etc., and externally applied movements (a car trip, a vibrator, arm band, wrist band, knee brace, heart rate monitor, etc.) is another option to power the sensor and preferably to charge a secondary power source such as an externally worn or attachable induction charging device.

Multiple sensors may be implemented to allow inter connectivity strings interconnected strands or strings of devices that may share data between sensors and react or modify activities in response to instructions from another member of the string or as pre-planned or modified by internal machine learning, e.g., to confirm or extend readings of interest. Individual members or a subset of members in a string may actively modulate the subject's health status. For example, an internal sensor may instruct another string component or data recipient to dispense a substance helpful to the subject. For example, if or when pre-migraine aura or an actual migraine is sensed, an appropriate chemical modifier may be released into the bloodstream or into cerebral spinal fluid. Deep brain stimulation may be carried out in response to sensor provided data and data analysis. Hormones, and paracrine signaling compounds are useful compounds in these regards.

The NSEs may themselves transmit or through a string transmit information (data) to external devices which may further share data to benefit from artificial intelligence protocols. The machine learning may aid in improving design but may also be a factor in instructions to the sensors and/or actuators. Preferred devices are self-diagnosing in a sense that signal strength is calibrated periodically to maintain stability of signal output as the device may age or change its deposition. The reporting between the sensors, their outputs and the main analytical processor thus is comparable over repeated measurements.

Actuators can serve a variety of tasks. An actuator may directly provide electrical stimulus to a target cell, a portion thereof, or a targeted area. An actuator may be stimulated to release a bio-active compound. An actuator may deliver a physical (kinetic) impulse to a targeted site. An actuator may be manipulated using a micro fiber including, but not limited to: carbon nanotube, polymers (e.g., nylon or other synthetic) preferably modified inertly to exhibit electricalmechanical traits, etc. Such micro fiber may itself participate in localized stimulus, but may mainly serve as a metering function, e.g., opening or closing a valve, providing pressure to deliver a substance from within an actuator component, and/or may act as a binary valve or as a valve variably metering delivery of a desired substance. A solenoid type actuator may be employed to control access of the deliverable substance to the targeted site. Delivery may involve simple diffusion when access to the body fluid is switched on or off or the area available for interaction is controlled. Delivery may be effected using a stored pressure. Delivery may be through pressure generated by action in the sensing/actuator device. The delivery may be in response to an external stimulus, a timed release (on a pre-programmed schedule or modulated by conscious or unconscious action of the host, an input from one or a plurality of sensors, a feedback response, etc.), a threshold signal responsive to sensor input, a signal from interplay between at least two sensor components—internal or external to the body, an instruction generated in accordance with an algorithm response to at least one internal sensor result, etc.

To avoid invasive surgery, a micro-implantation protocol is useful. Needleless injection can inject liquids such as vaccinations, but also microchips capable of marking location or confirming identity. A needleless or needle-free injection introduces a desired material into the body using an electromechanical pulse or intense pressure with high velocity to breach the skin for delivery of the desired bolus of material. The skin lesion is negligible, and self-adheres across the breach to, often in less than a second, close the transient breach. At present an antenna is frequently used to provide power to the microchip to initiate bidirectional communication. The length of the antenna frequently comprises the largest dimension of such injected device. However, pulsed electric field or mechanical stimulation, when constant or over an extended duration, can provide power independent of an antenna. Coiled antennae may themselves present as a longer receiver, but may reduce outside dimensions needed for device power and/or communication.

Device location is a primary consideration. Desired sensor location is dependent upon several factors including, but not limited to: type of data sought, source of data (organ, tissue, neurologic), ease of access, safety, intensity of available charging, suitable size capacity, subject impact, connectivity, cost, medical benefit, etc. Devices may be introduced into the body using any appropriate method including, but not limited to: a needle to precisely locate a device, may be introduced using a jetting process as common in needleless injection, may be electromagnetically or mechanically impulsed to a target site may be endoscopically delivered, etc. In some embodiments the force driving the sensor is terminated using a tether, that may remain as a path to given a length for the proper depth. Such use of tether the sensor for signaling or direct delivery of current or for removal when desired. For example, such sensor may be delivered to a specified depth, e.g., sub lipid, intramuscular, on a bone, etc., with the tether allowing precision delivery to a site of interest even when the mechanical properties of intervening tissues are imprecisely defined. Any non-immunogenic fiber of suitable strength and stability in the body can be considered as suitable. Such tether may be dissolvable or bio-absorbed.

Locating the device is an important consideration. The sensor must be in a location where the feature sensed is available. For example, in an area: where temperature is considered an important parameter, near a source of chemical to be assayed, actually participating in movement or change (e.g., changing pulse of a vessel, inflammation, air sac filling, intestinal mobility), etc. Accordingly, embodiments include near surface, i.e., subdermal applications, but also deeper, specific tissue or organ targeted devices. A CT or MRI scan are examples of tools that might be used to define, select and/or confirm a precise location for the device. In some tissues and where less precision is satisfactory, the scan or simple measurement might be used to calibrate a charge applied to the injected sensor to drive it to the desired site. A strand such as a carbon fiber can be used to limit length of travel when tissue inconsistencies would not allow precise location by charge alone.

In special circumstances where a barricade such as cartilage or bone provides a border defining a desired location, the acceptable charge may have a broad range, a slight excess over the minimum necessary to drive the sensor through the desired distance up to a significantly greater charge that is not unacceptably impactful to the sensor, the point of entry and the barricade upon which the sensor will rest.

The sensor unit itself is designed to meet the requirements for feature(s) to be reported. For example, a sensor may report the pulsing flow of blood, its timing, strength, and the like. The sensor may report temperature or other molecular movement. Biomolecules, dissolved ions, complexed metals, etc. are suitable targets for sensing. The sensor elements may be in contact with the sensed element through a selective membrane, e.g., permitting only passage of volatile organic components into a vapor phase chamber or liquid phase environment. The membrane may be in direct contact with the sensor element, and in several embodiments the sensor element itself may be exposed to the feature being monitored.

Reporting may be continuous, e.g., constant or at frequent intervals and may output data to a removable unit or extra-corporeal unit that can independently communicates with one or more devices. Such complementary unit is not constrained in size to a micro- or nano-meter size.

What is claimed is:

1. A sensing device attachable to or implantable within a human body, said device comprising:
a nano electronic component;
said nano electronic component comprising a communication interface;
said communication interface capable of communication with at least one component selected from the group consisting of: an actuator device, a sensor element and a data receiver;
said nano electronic component further comprising: an energy transponder able to transpond at least one form of energy selected from the group consisting of: kinetic motion, ionic gradient, chemical presence or concentration, humidity, and temperature gradient, to electric charge or current; and
said electric charge available to power at least one sensor element when implanted, said electric charge or current comprising a signal transmissible to a wired or remote data processor.

2. The sensing device of claim 1 with a diameter less than about 5×10⁻³ meters.

3. The sensing device of claim 1 with a diameter less than about 2×10⁻³ meters.

4. The sensing device of claim 1 with a diameter less than about 1.5×10⁻³ meters.

5. The sensing device of claim 1 with a diameter less than about 1.0×10⁻³ meters.

6. The sensing device of claim 1 with a diameter less than about 0.75×10⁻³ meters.

7. The sensing device of claim 1 with a diameter less than about 0.5×10⁻³ meters.

8. The sensing device of claim 1 further comprising a second energy transponder with a function able to transpond energy transmitted from a location exterior to the human body.

9. The sensing device of claim 8 wherein the energy transmitted from a location exterior to the human body is selected from the group consisting of: acoustic, light, alternating current, radio, vibration and magnetic.

10. The sensing device of claim 1 wherein at least one of said at least one sensor elements senses at least one characteristic selected from the group consisting of: [O₂], [CO₂], pH, concentration of a chemical messenger, temperature, alcohol, aldehyde, osmotic strength, ionic strength, rate of blood flow, pulse, pressure, movement, drug concentration and metabolite concentration.

11. The sensing device of claim 10 wherein said at least one of said at least one sensor elements senses concentration of at least one chemical messenger selected from the group consisting of: a hormone, a cytokine, an inhibitory factor, a releasing factor and a stimulating factor.

12. The sensing device of claim 11 wherein said at least one of said at least one sensor elements senses concentration of at least one chemical messenger selected from the group consisting of: adenosine, melatonin, insulin, adrenocorticotropic hormone (ADH), cortisol, luteinizing hormone, gonadotropin-releasing hormone (GRH), follicle stimulating hormone (FSH), dopamine, serotonin, thyroxin, triiodothyronine, epinephrine, norepinephrine, glucagon, monosodium glutamate (MSG), anti-müllerian hormone (AMH), adiponectin, endorphins, adrenocorticotropic hormone (ACTH), thyroid stimulating hormone (TSH), thyroid releasing hormone (TRH), cholecystokinin (CCK), gastric inhibitory polypeptide (GIP), corticotropin-releasing hormone (CRH), prolactin releasing hormone (PRH), bone gamma-carboxyglutamic acid-containing protein (BGLAP), glanin, renin, growth hormone, an endogenous cannabinoid, luteinizing hormone, oxytocin (OXT), and human chorionic gonadotropin (HCG).

13. The sensing device of claim 1 wherein at least one of said at least one sensor elements senses at least one characteristic selected from the group consisting of: a sugar concentration, glucose concentration, lipid concentration, volatilizable organic compounds, [Mg⁺²], [Ca⁺²] and at least one cannabinoid concentration.

14. The sensing device of claim 1 wherein at least one of said at least one sensor elements senses data relevant to at least one condition selected from the group consisting of: blood oxygenation, sleep cycling, inflammation, allergic responses, asthma, A-fib, ionic radiation, protein agglomeration, clotting, balance, pineal function, CNS function, a migraine, a seizure, pregnancy, fertility, esophageal function, muscle contraction and hiccups.

15. The sensing device of claim 14 further comprising an external component capable of providing an alert of a pre-symptomatic state associated with concentration of at least one chemical messenger selected from the group consisting of: a hormone, a cytokine, an inhibitory factor, a releasing factor and a stimulating factor.

16. The sensing device of claim 14 further comprising an actuation component that upon detecting an alert condition relevant to at least one chemical messenger selected from the group consisting of: a hormone, a cytokine, an inhibitory factor, a releasing factor and a stimulating factor has capability to prevent, modulate or slow development of said condition.

17. The sensing device of claim 14 wherein said capability is under direction of a machine learning process.

18. The sensing device of claim 1 wherein said sensor element comprises a nanosensor element.

19. The sensing device of claim 18 wherein said nanosensor element is carbon based.

20. The sensing device of claim 18 wherein said nanosensor element comprises at least one element selected from the group consisting of: Si, B, Ga, C, N and Al.

21. The sensing device of claim 5 wherein said second energy transponder also transmits instruction or data.

22. The sensing device of claim 1 wherein said communication interface employs data encryption.

23. The device of claim 1 wherein said communication interface comprises a geolocation application.

24. The device of claim 23 wherein said geolocation application alerts a designated person or authority when a subject is absent from a designated location.

25. The device of claim 23 wherein said geolocation application reports said location to a designated person or authority.

26. The device of claim 1 comprising an interface that provides a medical alert when a predetermined criterion is met.
